(19) United States
(12) Patent Application Publication
(10) Pub. No.: US 2006/0161106 A1
(43) Pub. Date: Jul. 20, 2006

(54) DISPOSABLE RETRACTABLE SAFETY SYRINGE HAVING A HOLLOW TRUNCATED CONE WITH A SLIT

(76) Inventor: Peng-Chieh Wu, Taipei (TW)

Correspondence Address: NORTH AMERICA INTELLECTUAL PROPERTY CORPORATION, P.O. BOX 506, MERRIFIELD, VA, 22116, US

(21) Appl. No.: 10/904,419

(22) Filed: Nov. 9, 2004

(51) Int. Cl. A61M 5/00; A61M 5/32

(52) U.S. Cl. 604/110; 604/195

(57) ABSTRACT

A retractable safety syringe includes a syringe barrel, a needle mount, a plunger rod and a needle. The needle mount includes a hollow truncated cone extending inward and upward with a completely surrounded lower conical portion. The hollow truncated cone includes a slit cutting from a ridge of the truncated cone. The plunger rod includes a rod and a plunger formed at one end of the rod. The plunger has a plunger crown and a detent connected to the plunger crown. The hollow truncated cone containing a slit allows the plunger rod's detent to latch on to the needle mount making the plunger rod and needle mount components become jointly retractable. Using the hollow truncated cone design augments the robustness factor of the needle retraction mechanism, using the slit increases its ease of use, while both hollow truncated cone and slit jointly minimize fluid loss.

### BACKGROUND OF INVENTION

1. Field of the Invention

The present invention relates to a disposable safety syringe, and more particularly, to a disposable safety syringe whose needle is retracted after injection to prevent reuse and needle stick injuries.

2. Description of the Prior Art

Due to the serious risk of contamination to various diseases by using infected needles, virtually all syringes in use nowadays are disposable. Furthermore, some of these also possess a retractable mechanism, which retracts the needle inside the syringe after use so that the person administering the injection is prevented from impaling himself and others with a potentially infected needle.

Accordingly, the needle retraction mechanism is considered an especially critical component. Issues such as minimizing the quantity of medicinal fluid being trapped inside the syringe after needle retraction, the amount of pressure required to engage the hooking mechanism and the robustness of the hooking mechanism itself are essential to the creation of an excellent needle retraction mechanism.

Please refer to FIG. 1, where all the essential components of a retractable safety syringe 150 are illustrated. First, a needle 100 comprises of a needle seat 102 that connects to a needle cannula 104. Secondly, a syringe barrel 110 comprises at a lower end a winged handle 112, and at an upper end, a socket 108, containing a cavity 106. Thirdly, a needle mount 114 comprises at an upper end a needle holder 116 and at a lower end, assembled on the inside walls of the needle mount 114, a clamp 118 and a clamp 118′. The clamps 118 and 118′ are positioned symmetrically with respect to each other. Finally, a plunger rod 134 comprises at an upper end a hooking mechanism 120, which is made up of a stem 124 that terminates in a detent 122. The hooking mechanism 120 is connected to a crown 126, which is the terminating upper layer of a plunger 128. The plunger 128 is further connected to a rod 130, which terminates at a lower end with a thumb press 132.

Please refer to FIG. 2. FIG. 2 is a magnified view of the needle mount 114 shown in FIG. 1. In addition to the needle holder 116 and the clamps 118 and 118′, the needle mount 114 comprises a plug section 200, which allows the needle mount to be attached at assembly time air tightly inside the syringe barrel 110, and a pulling member 202, which is the lower part of the needle mount 114 containing the clamps 118 and 118′.

Note that the hooking mechanism's 120 detent 122 and stem 124, the pulling member 202 and the clamps 118 and 118′ constitute the needle retraction mechanism.

The needle retraction mechanism becomes engaged when the plunger rod is fully inserted inside the syringe barrel by additional pressure on the thumb press, so that the hooking mechanism goes passed the clamps and the detent latches on to them.

This type of needle retraction mechanism, which uses the two clamps 118 and 118′, has two serious shortcomings. First of all, the clamps 118 and 118′ are protruding components that are latched on to the wall of pulling member 202. As such, they are not sufficiently robust and can, henceforth, as protrusions can generally break easily. Secondly, using the hooking mechanism 120, as suggested by prior art, does not minimize the loss of medicinal fluid. While it is true that the space between the clamps 118 and 118′ allows the free passage of fluid, this does not prevent fluid to be caught that between the detent 122 and plunger crown 126 after the detent 122 has latched on to the clamps 118 and 118′, as the hooking mechanism 120 does not have the ability expel fluid trapped between itself and the crown 126.

Please refer to FIG. 3 where all the essential components of another retractable safety syringe 350 are illustrated. First, a needle 300 is illustrated. Secondly, a syringe barrel 304 comprises at an upper end a retaining edge 302, and at a lower end, a winged handle 306. Thirdly, a needle mount 310 comprises an inner hub 308, a retaining groove 312 and a ring 314, which is fastened inside the needle mount 310. The needle mount 310 is air tightly installed inside the syringe barrel 304, at its upper end, so that the syringe barrel's 304 retaining edge 302 retains the needle mount's 310 retaining groove 312. Finally, a plunger rod 328 comprises at an upper end a hooking mechanism 320, which is made up of a stem 318 that terminates in a detent 316. The hooking mechanism 320 is connected to a crown 322, which is the terminating upper layer of a plunger 324. The plunger 324 is further connected to a rod 326, which terminates at a lower end with a thumb press 340. Note that the hooking mechanism's 320 detent 316 and stem 318, plus the ring 314 constitute the needle retraction mechanism.

Please refer to FIG. 4. FIG. 4 is a magnified view of the needle mount's 310 ring 314, shown in FIG. 3. The ring 314 comprises a ring face 402 and a central hollow 400 in the middle of the ring 314.

This kind of hooking mechanism has its own drawbacks, which are as follows. First of all, in order for the detent 316 to go through the central hollow 400, an unreasonable amount of pressure is required to push the plunger. Consequently, it is probable that the individual administering the injection does not have the sufficient strength to generate sufficient pressure on the thumb press in order to get the detent through the central hollow 400 of the ring 314. Furthermore, the large force applied on the plunger will cause the syringe and the needle to shake while the needle is still in the patient, which is dangerous and uncomfortable for the patient.

Another disadvantage of the prior art is that this type of hooking mechanism does not minimize fluid loss to the maximum extent possible. As the detent 316 latches on to the ring 314, some fluid becomes trapped below the face 402 of the ring 314, between the face 402 and the plunger crown 322. This occurs due to the fact that the neck 318 acts as an obstacle that prevents the face 402 of the ring 314 from touching the crown 322, hence creating an area that will be filled with fluid even after the hooking mechanism has been engaged.

### SUMMARY OF INVENTION

It is therefore a primary objective of the claimed invention to provide a disposable retractable safety syringe that solves the above-mentioned problems of the prior art.

According to the claimed invention, a retractable safety syringe comprises a syringe barrel, a needle mount, a plunger rod and a needle. The needle mount is assembled at an upper end of the syringe barrel and formed with an inner hub, and a hollow truncated cone extending inward and upward with a completely surrounded lower conical portion. The hollow truncated cone includes a slit cutting from a ridge of the truncated cone. The plunger rod includes a rod and a plunger formed at one end of the rod. The plunger has a plunger crown and a detent connected to the plunger crown. The needle includes a needle cannula coupled to an upper portion of the inner hub.

These and other objectives of the present invention will no doubt become obvious to those of ordinary skill in the art after reading the following detailed description of the preferred embodiment that is illustrated in the various figures and drawings.

### BRIEF DESCRIPTION OF DRAWINGS

FIG. 1 is an exploded perspective view of a prior art retractable safety syringe.

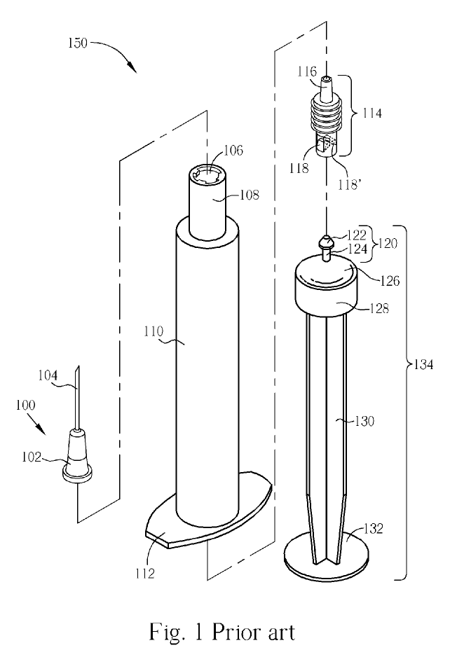

FIG. 2 is a perspective view of the needle mount of the prior art retractable safety syringe in FIG. 1.

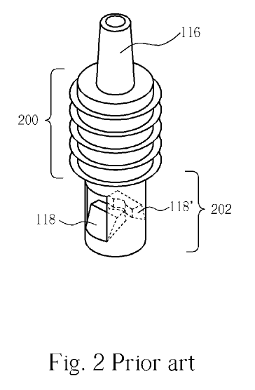

FIG. 3 is an exploded perspective view of another prior art retractable safety syringe.

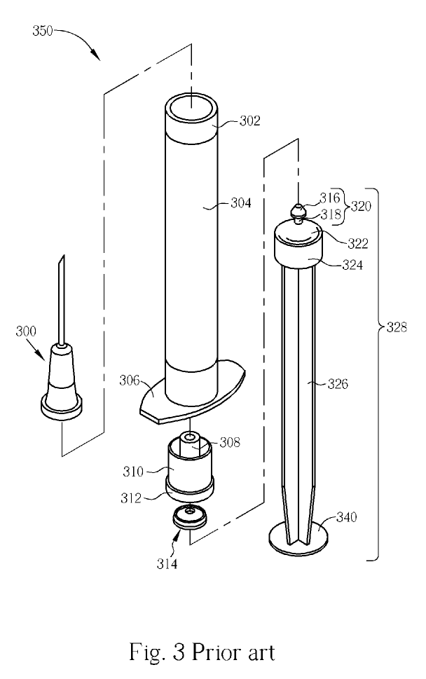

FIG. 4 is a perspective view of the needle mount's connection ring of the retractable safety syringe in FIG. 3.

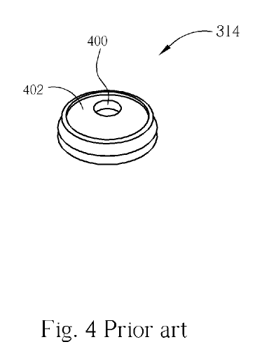

FIG. 5 is an exploded perspective view of a first retractable safety syringe according to the current invention.

FIG. 6 is a perspective view of the needle mount of the retractable safety syringe in FIG. 5.

FIG. 7 is a perspective view of the plunger rod of the retractable safety syringe in FIG. 5.

FIG. 8 is an exploded perspective view of a second retractable safety syringe according to the current invention.

FIG. 9 is a perspective view of the plunger rod of the retractable safety syringe in FIG. 8.

FIG. 10 is a schematic operation view of the retractable safety syringe in FIG. 5 before the hooking mechanism is engaged.

FIG. 11 is a schematic operation view of the retractable safety syringe in FIG. 5 after the hooking mechanism is engaged.

FIG. 12 is a schematic operation view of the retractable safety syringe in FIG. 5 after the retraction of the needle mount has commenced.

FIG. 13 is a perspective view of another needle mount of the retractable safety syringe in FIG. 5.

### DETAILED DESCRIPTION

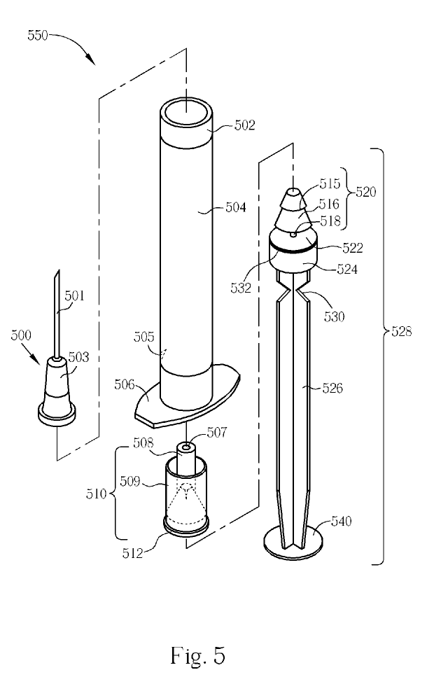

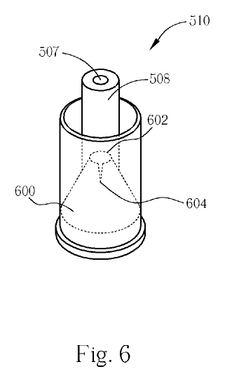

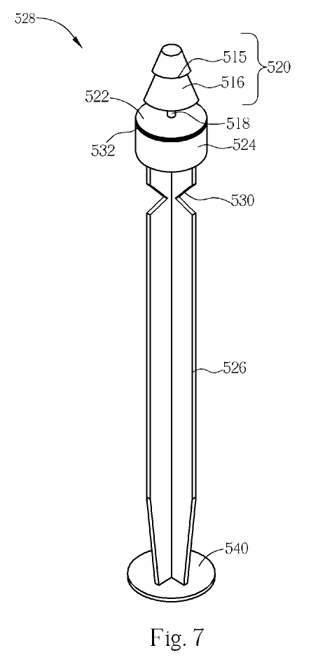

Please refer to FIG. 5 in conjunction with FIG. 6 and FIG. 7, for a first embodiment of a disposable retractable safety syringe 550 according to the present invention. First, the retractable safety syringe 550 comprises a needle 500, which includes a cannula 501 that is connected to an upper portion of an inner hub 508. It is possible for the cannula 501 to be coupled to the upper portion of the inner hub 508 via a needle base 503. Secondly, a syringe barrel 504 comprises at an upper end a retaining edge 502, and at a lower end, a winged handle 506. The syringe barrel 504 also comprises on its interior a stopper 505, which is used to allow a plunger rod 528 to be inserted into the syringe barrel 504 and prevents a needle mount 510 from being removed from the syringe barrel 504. The stopper 505 could be in the form of a flange or a rim, continuously or discontinuously formed on the inside of the syringe barrel 504.

Subsequently, the needle mount 510 comprises the inner hub 508, an outer hub 509, a fluid passage 507 and a retaining groove 512. The needle mount 510 is air tightly installed inside the syringe barrel 504, at its upper end, so that the syringe barrel's 504 retaining edge 502 retains the needle mount's 510 retaining groove 512, preventing the needle mount 510 from coming out at the upper end of the syringe barrel 504. It is to be noted that the outer hub 509 is an optional element, meaning that the retractable safety syringe 550 could be manufactured with or without an outer hub.

As shown in FIG. 6, the needle mount 510 further comprises a hollow truncated cone 600, which has its base attached to the inside of the needle mount 510 and could be made of an elastic material. The hollow truncated cone 600 includes at its upper opening a ridge 602. Also forming at the hollow truncated cone's 600 upper opening there is a slit 604. Although just one slit is disclosed in FIGS. 5 and 6, the hollow truncated cone 600 may contain more than one slit in this embodiment. This invention is not limited to only one slit.

Furthermore, a plunger rod 528 comprises at an upper end a detent 520, which is made up of a connector 518 that continues with a truncated cone-shaped neck 516. The truncated cone-shaped neck 516 contains on its upper end a detent lip 515. The detent 520 is connected to a crown 522, which is the terminating upper layer of a plunger 524. The plunger 524 contains on its upper end a color band 532 that serves for identification of the fluid level in the syringe barrel 504. The plunger 524 is further connected to a rod 526, which terminates at a lower end with a thumb press 540. The rod 526 further comprises a V-notch 530 that facilitates the breaking of the rod 526 after use, for the purpose of recycling the rod 526 separately from the rest of the syringe and preventing the needle from being pushed out of the injecting end of the syringe barrel 504 after retraction.

Note that the detent's 520 truncated cone-shaped neck 516, detent lip 515, connector 518, plus the needle mount's 510 hollow truncated cone 600 constitute the needle retraction mechanism.

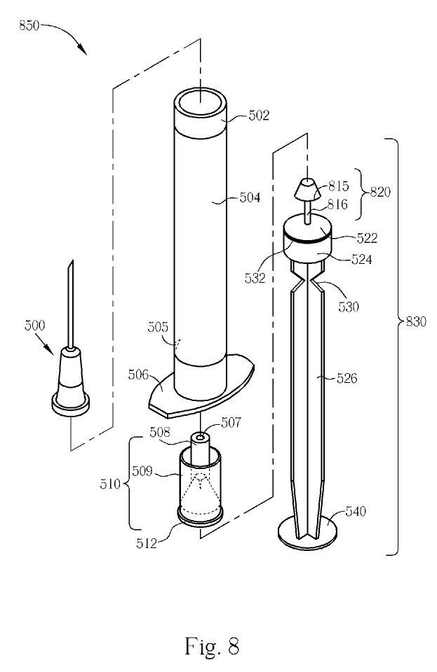

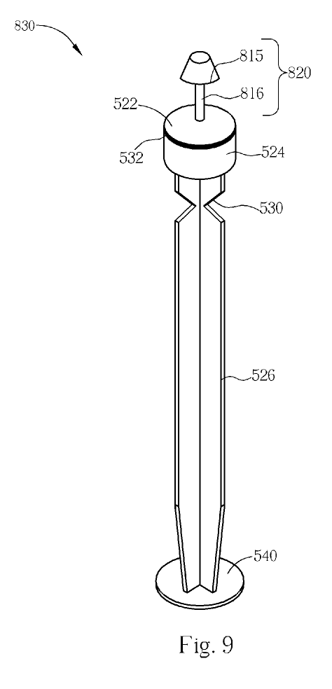

Please refer to FIG. 8 in conjunction with FIG. 6 and FIG. 9, for a second embodiment of a disposable retractable safety syringe 850 according to the present invention. The retractable safety syringe 850 comprises a needle 500, a syringe barrel 504, a needle mount 510 and a plunger rod 830. It is to be noted that the needle 500, syringe barrel 504 and needle mount 510 are identical to the ones illustrated in FIGS. 5 and 6.

The difference between the retractable safety syringes 850 and 550 is in the structure of the plunger rods 830 and 528. The plunger rod 830 of the retractable safety syringe 850 comprises at an upper end a detent 820, which is made up of a connector 816 and a detent lip 815. The detent 820 is connected to a crown 522, which is the terminating upper layer of a plunger 524. The plunger 524 contains on its upper end a color band 532. The plunger 524 is further connected to a rod 526, which terminates at a lower end with a thumb press 540. The rod 526 further comprises a V-notch 530.

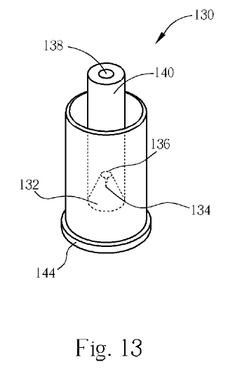

Please refer to FIG. 13 which illustrates another embodiment of a needle mount 130 of the retractable safety syringe 550, 850. Please refer to FIG. 13 in conjunction with FIG. 5 and FIG. 6 as an example.

A needle mount 130 comprises an inner hub 140 and a retaining groove 144. The inner hub 140 includes a fluid passage 138 and a hollow truncated cone 132, which has its base attached to the inside of the inner hub 140. The hollow truncated cone 132 includes at its upper opening a ridge 136. Also forming at the hollow truncated cone's 132 upper opening there is one or more slits 134.

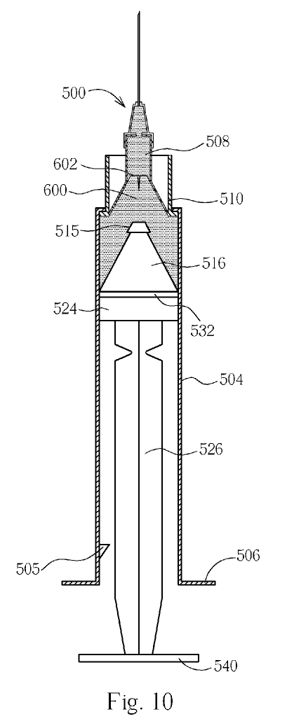

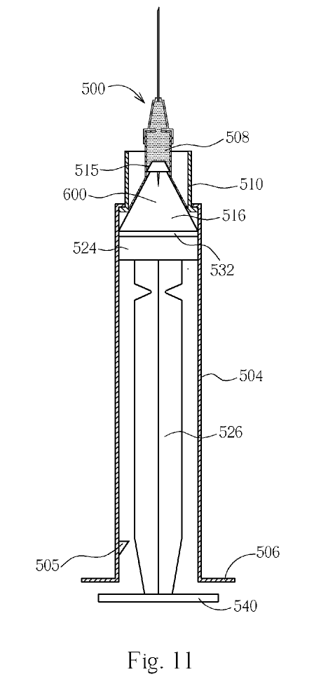

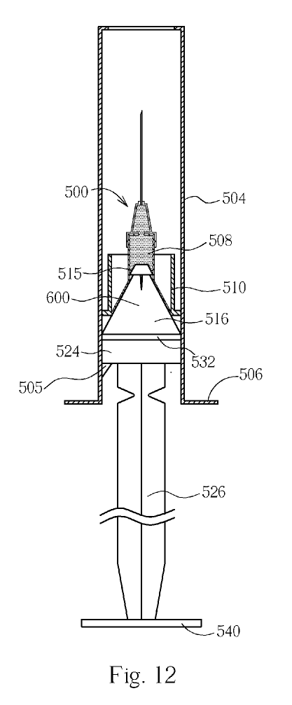

Please refer to FIG. 10, FIG. 11 and FIG. 12 which are schematic operational views of the fully assembled retractable safety syringe at three points in time: before, at the exact time and after the syringe's hooking mechanism has been engaged. Please refer to these figures in conjunction with FIGS. 5, 6 and 7.

First of all, in FIG. 10 it can be seen that the rod 526 has not been fully inserted in the syringe barrel 504, and hence the detent lip 515 have not yet latched onto the hollow truncated cone's 600 ridge 602. The gray area of FIG. 10 represents the area of the syringe that still contains medicinal fluid. Subsequently, in FIG. 11, the exact instance when the detent 520 is engaged is displayed. Here, the rod 526 is fully inserted in the syringe barrel 504, and consequently, the detent lip 515 has penetrated past the ridge 602, causing the plunger rod 830 and the needle mount 510 to ‘hook’. At this point, the downward movement of the plunger rod 830 would dislodge the needle mount 510. It is to be noted that in FIG. 11, the gray area representing the fluid remaining in the syringe is smaller than in FIG. 10 as engaging the hooking mechanism has caused almost all of the fluid in the syringe to be expelled. Finally, FIG. 12 illustrates an instance when the needle mount 510 has been retracted from its original position in the upper end of the syringe barrel 504 and the needle 500 itself is inside the syringe barrel 504. At this point, since the needle mount 510 is no longer air tightly attached to the syringe barrel 504, any movement would cause plunger rod 830, the needle mount 510 and hence the needle 500 to move in unison. Preventing the needle mount 510 to be drawn out of the syringe barrel 504 on its lower end is the stopper 505, which stops the plunger 524 from going past it, and consequently the needle 500, needle mount 510 and plunger rod 528 from exiting the syringe barrel 504 at the lower end. Furthermore, the combination of needle 500, needle mount 510 and plunger rod 830 are prevented from exiting the syringe barrel 504 at its upper end by the retaining edge 502.

Minimizing the quantity of medicinal fluid being trapped inside the syringe after needle retraction and reducing the amount of pressure required to engage the hooking mechanism while increasing the robustness of the hooking mechanism are issues not adequately addressed by the prior art. However, the present invention manages to address and to solve many of these problems that were left unsolved in prior art retractable safety syringes.

First of all, as it has been discussed above, the prior art has not been able to minimize the amount of the fluid trapped in the syringe to the same degree as the present invention. In the present invention, due to the shape of the detent, which hermetically hugs the inside of the hollow truncated cone as the hooking mechanism is engaged, the amount of little fluid being trapped is insignificant, since being only limited to small portions of the inner hub of the needle mount. However, in the prior art, fluid is also being caught between the plunger crown and the detent itself, making this aspect of the prior art inferior to the current invention.

Also different from the prior art, the present invention is successful in maximizing the robustness of the hooking mechanism, while at the same time minimizing the pressure required to drive the detent lip past the hollow truncated cone's upper opening ridge.

Robustness is primarily attained by using the hollow truncated cone, which unlike the prior art's clamps or ring, is inherently a sturdier shape that is capable to bear more pressure on its upper opening than a mechanism employing either a ring or clamps.

Moreover, in the present invention, the pressure required to engage the hooking mechanism is not unreasonable, and fortunately, it does not come at the expense of robustness. To ensure that everybody can engage the syringe's hooking mechanism without undue effort and to prevent excessive movement of the needle while it is still inside the patients body, the present invention has procured the hollow truncated cone with a slit. The slit permits the upper opening of the hollow truncated cone to expand more readily than it would be possible if there was no slit and the detent lip would have had to force its way through a rigid upper opening of the hollow truncated cone.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A retractable safety syringe comprising:
a syringe barrel;
a needle mount assembled at an upper end of the syringe barrel and formed with an inner hub, and a hollow truncated cone extending inward and upward with a completely surrounded lower conical portion, the hollow truncated cone including a slit cutting from a ridge of the truncated cone;
a plunger rod including a rod and a plunger formed at one end of the rod, the plunger having a plunger crown and a detent connected to the plunger crown; and
a needle including a needle cannula coupled to an upper portion of the inner hub.

2. The retractable safety syringe of claim 1 wherein the detent comprises a connector connected to the plunger crown, and a truncated cone-shaped neck connected to the connector, the neck having a detent lip for hooking on a ridge of the hollow truncated cone.

3. The retractable safety syringe of claim 1 wherein the detent comprises a connector connected to the plunger crown, and a detent lip for hooking on a ridge of the hollow truncated cone.

4. The retractable safety syringe of claim 1 wherein the syringe barrel comprises a winged handle formed at a lower end of the syringe barrel.

5. The retractable safety syringe of claim 1 wherein the needle mount further comprises an outer hub encasing the inner hub and the hollow truncated cone.

6. The retractable safety syringe of claim 1 wherein the hollow truncated cone is attached to the outer hub.

7. The retractable safety syringe of claim 1 wherein the hollow truncated cone is attached to the inner hub.

8. The retractable safety syringe of claim 1 wherein the plunger rod contains a V-notch.

9. The retractable safety syringe of claim 1 wherein the hollow truncated cone is made of an elastic material.

10. The retractable safety syringe of claim 1 wherein the syringe barrel has a stopper close to a lower end of the syringe barrel.

11. The retractable safety syringe of claim 1 wherein a color band of contrasting color is assembled around the plunger.

12. The retractable safety syringe of claim 1 wherein the needle further includes a needle base connected to the upper portion of the inner hub, and the needle cannula is mounted on the needle base.

13. The retractable safety syringe of claim 1 wherein the needle cannula is connected to the upper portion of the inner hub.

14. A needle mount of a safety syringe comprising:
a hollow truncated cone extending inward and upward with a completely surrounded lower conical surface, the hollow truncated cone including a slit cutting from a top edge of the truncated cone; and
an inner hub encasing an upper portion of the hollow truncated cone.

15. The needle mount of claim 14 further comprising an outer hub encasing the inner hub and the hollow truncated cone.

16. The needle mount of claim 14 wherein the hollow truncated cone is attached to the outer hub.

17. The needle mount of claim 16 wherein a lower end of the hollow truncated cone is attached to the outer hub.

18. The needle mount of claim 14 wherein the hollow truncated cone is attached to the inner hub.

19. The needle mount of claim 18 wherein a lower end of the hollow truncated cone is attached to the inner hub.

20. A plunger rod of a safety syringe comprising:
a rod; and
a plunger formed at one end of the rod, the plunger including:
a plunger crown; and
a detent connected to the plunger crown, the detent including a truncated cone-shaped neck having a detent lip for hooking.

21. The plunger of claim 20 wherein the detent further comprises a connector connected between the plunger crown and the truncated cone-shaped neck.
